(19) United States
(12) Patent Application Publication
(10) Pub. No.: US 2024/0324997 A1
(43) Pub. Date: Oct. 3, 2024

(54) Ultrasonic Probe

(71) Applicant: SEIKO EPSON CORPORATION, Tokyo (JP)

(72) Inventors: Keita KUBO, Matsumoto (JP); Chikara KOJIMA, Matsumoto (JP); Hikaru IWAI, Matsukawa (JP); Eiji OSAWA, Matsumoto (JP)

(21) Appl. No.: 18/619,391

(22) Filed: Mar. 28, 2024

(30) Foreign Application Priority Data
Mar. 29, 2023 (JP) 2023-052915

(51) Int. Cl. A61B 8/00; G01S 7/52; G01S 15/89

(52) U.S. Cl. CPC A61B 8/4494; A61B 8/4444; G01S 7/52079; G01S 15/8925

(57) ABSTRACT

An ultrasonic probe includes: an ultrasonic device configured to transmit an ultrasonic wave; and an acoustic sheet provided at a transmission surface of the ultrasonic wave of the ultrasonic device. The acoustic sheet includes an acoustic lens configured to guide the ultrasonic wave output from the transmission surface in a predetermined direction, a base material covering at least a part of the acoustic lens and having a difference in acoustic impedance from a measurement target less than a predetermined threshold, and a first fixing portion provided at least at a first surface of the base material and configured to detachably fix the acoustic sheet to the measurement target, the first surface being configured to face the measurement target.

The present application is based on, and claims priority from JP Application Serial Number 2023-052915, filed Mar. 29, 2023, the disclosure of which is hereby incorporated by reference herein in its entirety.

### BACKGROUND

### 1. Technical Field

The present disclosure relates to an ultrasonic probe.

### 2. Related Art

In the related art, an ultrasonic probe that transmits and receives ultrasonic waves to and from a living body is known (for example, see JP-A-2019-72369).

JP-A-2019-72369 includes an ultrasonic probe (ultrasonic diagnostic apparatus) in which a sensor including a vibrator and an acoustic lens for transmitting the ultrasonic waves is incorporated, and a cover (ultrasonic diagnostic apparatus cover) for preventing adhesion of blood or the like to the ultrasonic probe. The cover includes a soft bag-shaped tubular body into which the ultrasonic probe is inserted, an inner bracket that guides the insertion of the ultrasonic probe into the tubular body, and a probe cover body that covers the sensor of the ultrasonic probe.

In the ultrasonic probe disclosed in JP-A-2019-72369, the sensor is provided at a distal end of a tubular casing, and an operator holds the sensor by bringing the sensor into contact with a predetermined position of the living body with a hand.

In contrast, in a thin ultrasonic probe that is provided with a piezoelectric element at a vibrating membrane and outputs ultrasonic waves by driving the piezoelectric element and vibrating the vibrating membrane, the apparatus itself can be made thinner, smaller, and lighter and can be used by attaching it to a living body. In such a thin ultrasonic probe, a propagation efficiency of the ultrasonic waves can be made higher than that of the ultrasonic probe disclosed in JP-A-2019-72369.

JP-A-2019-72369 is an example of the related art.

When the ultrasonic probe is inserted into the cover as in JP-A-2019-72369, there is a high possibility that an air layer is interposed therebetween, and the propagation efficiency of the ultrasonic waves is reduced due to reflection of the ultrasonic waves in the air layer. In addition, when the cover in JP-A-2019-72369 is applied to the thin ultrasonic probe as described above, a resin plate is interposed between the ultrasonic probe and the living body. Therefore, the propagation efficiency of the ultrasonic waves further decreases due to a difference in acoustic impedance.

In particular, the thin ultrasonic probe that outputs the ultrasonic waves by vibrating the vibrating membrane by the piezoelectric element has a higher propagation efficiency of the ultrasonic waves to a measurement target than a bulk-type ultrasonic probe that vibrates a piezoelectric body itself. When the propagation efficiency of the ultrasonic waves is reduced due to the interposition of the resin plate as described above, advantages of the thin ultrasonic probe will be lost.

### SUMMARY

An ultrasonic probe of a first aspect according to the present disclosure includes: an ultrasonic device configured to transmit an ultrasonic wave; and an acoustic sheet provided at a transmission surface of the ultrasonic wave of the ultrasonic device, in which the acoustic sheet includes an acoustic lens configured to guide the ultrasonic wave output from the transmission surface in a predetermined direction, a base material covering at least a part of the acoustic lens and having a difference in acoustic impedance with the measurement target less than a predetermined threshold, and a first fixing portion provided at least at a first surface of the base material and configured to detachably fix the ultrasonic device to the measurement target, the first surface being configured to face the measurement target.

### BRIEF DESCRIPTION OF THE DRAWINGS

FIG. 1 is a perspective view schematically illustrating an ultrasonic probe according to a first embodiment.

FIG. 2 is a schematic cross-sectional view of the ultrasonic probe taken along a plane A in FIG. 1.

FIG. 3 is a schematic plan view illustrating an example of an ultrasonic element according to the first embodiment.

FIG. 4 is a schematic cross-sectional view of the ultrasonic element taken along a line B-B in FIG. 3.

FIG. 5 is a diagram illustrating a correlation between a ratio Z₁₂(=Z₂/Z₁) of an acoustic impedance and a sound intensity reflectance RI.

FIG. 6 is a cross-sectional view schematically illustrating a schematic configuration of an ultrasonic probe according to a second embodiment.

FIG. 7 is a cross-sectional view schematically illustrating a schematic configuration of another ultrasonic probe according to the second embodiment.

FIG. 8 is a cross-sectional view schematically illustrating a schematic configuration of an ultrasonic probe according to a third embodiment.

FIG. 9 is a cross-sectional view schematically illustrating a schematic configuration of another ultrasonic probe according to the third embodiment.

FIG. 10 is a cross-sectional view schematically illustrating a schematic configuration of an ultrasonic probe according to Modification 1.

FIG. 11 is a cross-sectional view schematically illustrating a schematic configuration of another ultrasonic probe according to Modification 1.

FIG. 12 is a cross-sectional view schematically illustrating a schematic configuration of still another ultrasonic probe according to Modification 1.

FIG. 13 is a plan view of an ultrasonic probe according to Modification 5 as viewed from a Z direction.

### DESCRIPTION OF EMBODIMENTS

### First Embodiment

Hereinafter, a first embodiment of the present disclosure will be described.

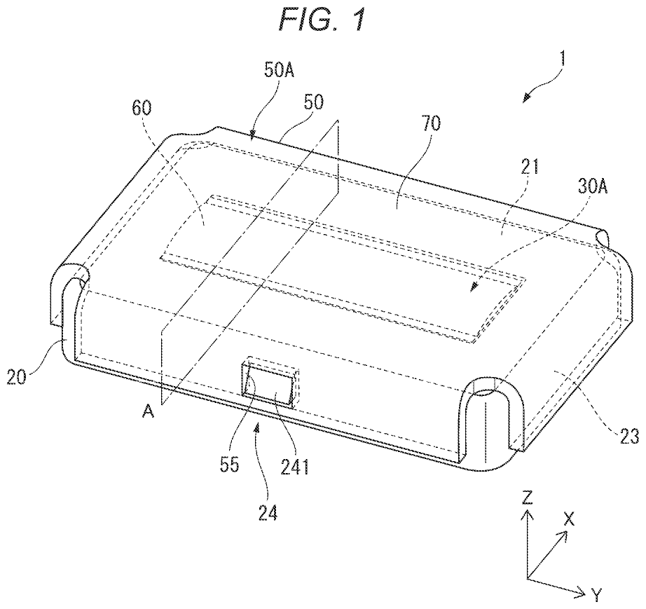

FIG. 1 is a perspective view of an ultrasonic probe 1 according to the first embodiment.

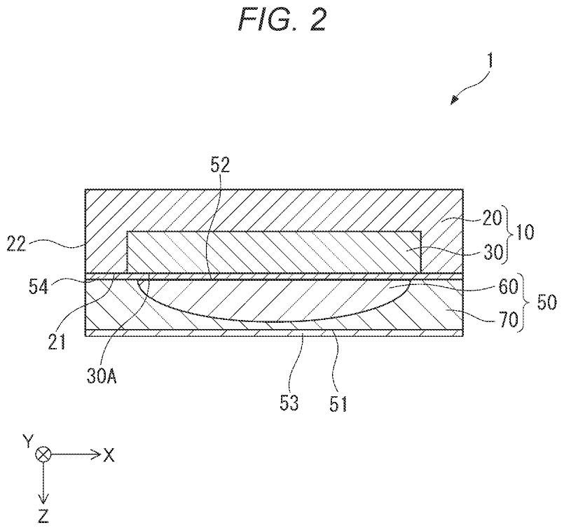

FIG. 2 is a schematic cross-sectional view of the ultrasonic probe 1 in FIG. 1 taken along a plane A.

As illustrated in FIGS. 1 and 2, the ultrasonic probe 1 of the embodiment includes an ultrasonic device 10 and a fixing sheet (acoustic sheet) 50. Then, by attaching the ultrasonic device 10 to a measurement target such as a living body by using the fixing sheet 50, the ultrasonic probe 1 is detachably fixed to the measurement target.

### Configuration of Ultrasonic Device 10

As illustrated in FIG. 2, the ultrasonic device 10 includes a housing 20 and an ultrasonic element 30 accommodated in the housing 20. FIG. 2 schematically illustrates a schematic configuration of the ultrasonic device 10, and a thickness of the ultrasonic element 30 with respect to the housing 20 is different from an actual thickness. Although not illustrated in FIG. 2, the housing 20 may include a fixing mechanism that fixes the ultrasonic element 30, a control board that controls the ultrasonic element 30, a battery that supplies electric power, and the like, and arrangement spaces may also be provided for accommodating the members.

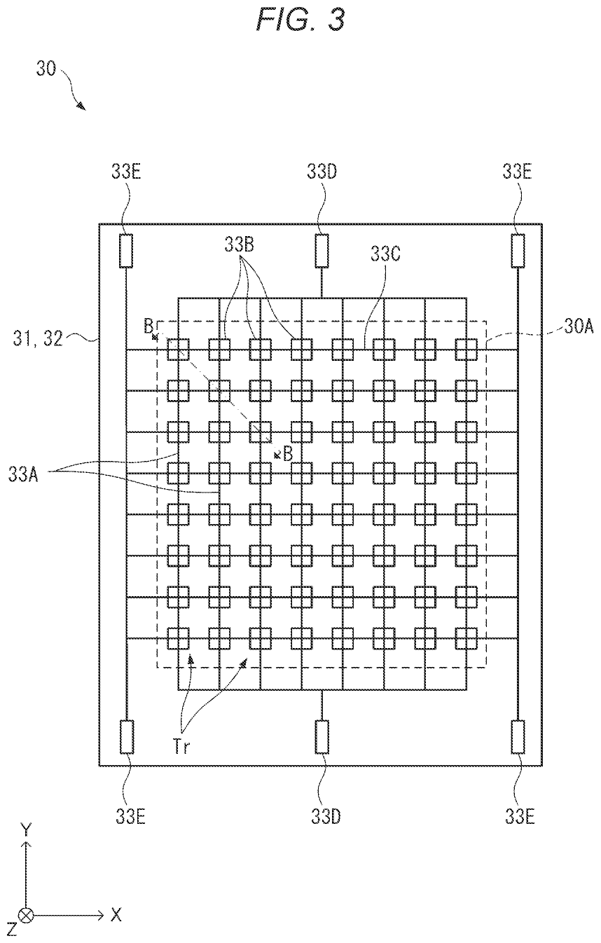

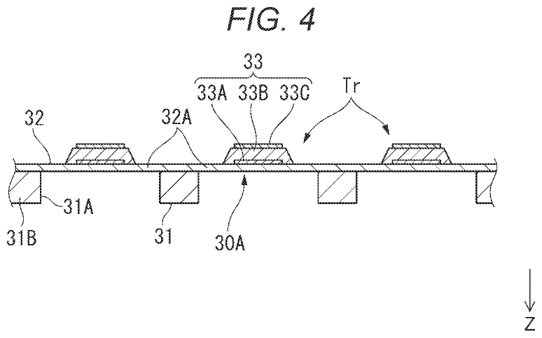

FIG. 3 is a plan view illustrating an example of the ultrasonic element 30 according to the embodiment. FIG. 4 is a schematic cross-sectional view of the ultrasonic element 30 taken along a line B-B in FIG. 3.

In the ultrasonic element 30, a plurality of ultrasonic transducers Tr are disposed in a two-dimensional array shape along an X direction and a Y direction. Here, a direction orthogonal to the X direction and the Y direction corresponds to a Z direction. The Z direction corresponds to a transmission direction of ultrasonic waves of the present disclosure in which the ultrasonic waves are transmitted.

Although the number of the disposed ultrasonic transducers Tr is reduced in FIG. 3 for convenience of description, actually, more ultrasonic transducers Tr may be disposed.

As illustrated in FIG. 4, the ultrasonic element 30 includes an element substrate 31, a vibrating plate 32 provided above the element substrate 31, and piezoelectric elements 33 provided above the vibrating plate 32.

The element substrate 31 is implemented by a semiconductor substrate such as Si. Substrate openings 31A corresponding to the respective ultrasonic transducers Tr are provided in the element substrate 31. In the embodiment, each substrate opening 31A is a through hole running in a substrate thickness direction (Z direction) of the element substrate 31, and the vibrating plate 32 is provided on a-Z side of the through hole.

A side (+Z side) of the substrate opening 31A on which the vibrating plate 32 is not provided may be filled with an acoustic matching material having an acoustic impedance close to the living body which is the measurement target. As the acoustic matching material, a resin material such as silicone can be used.

The vibrating plate 32 is implemented by, for example, a stacked body of SiO₂ and ZrO₂, and is provided to cover an entire-Z side of the element substrate 31. That is, the vibrating plate 32 is supported by a partition wall 31B constituting the substrate opening 31A, and closes the −Z side of the substrate opening 31A. A thickness dimension of the vibrating plate 32 is sufficiently smaller than that of the element substrate 31.

The piezoelectric elements 33 are provided above the vibrating plate 32 that closes the substrate openings 31A. The piezoelectric element 33 is implemented by, for example, a stacked body in which a lower electrode 33A, a piezoelectric film 33B, and an upper electrode 33C are stacked from the vibrating plate 32 toward the −Z side.

Here, a portion of the vibrating plate 32 that closes the substrate opening 31A constitutes a vibrating portion 32A, and one ultrasonic transducer Tr is implemented by the vibrating portion 32A and the piezoelectric element 33.

In such an ultrasonic transducer Tr, when a rectangular wave (driving voltage signal) having a predetermined frequency is applied between the lower electrode 33A and the upper electrode 33C, the piezoelectric film 33B is bent, the vibrating portion 32A vibrates, and the ultrasonic waves are transmitted to the +Z side. Further, when the vibrating portion 32A vibrates by ultrasonic waves (reflected waves) reflected from the living body, a potential difference occurs between upper and lower portions of the piezoelectric film 33B. Accordingly, by detecting the potential difference generated between the lower electrode 33A and the upper electrode 33C, the received ultrasonic waves can be detected.

In the embodiment, as illustrated in FIG. 3, the lower electrodes 33A of the ultrasonic transducers Tr arranged in the Y direction are coupled to one another. The lower electrodes 33A are coupled to drive terminals 33D, and are electrically coupled from the drive terminals 33D to a control board (not illustrated) via, for example, a flexible printed circuit board.

Further, the upper electrodes 33C of the ultrasonic transducers Tr arranged in the X direction are coupled to one another. The upper electrodes 33C are coupled to common terminals 33E. The common terminals 33E are electrically coupled to the control board via, for example, a flexible printed circuit board.

Here, in the embodiment, since a traveling direction of the ultrasonic waves is controlled by an acoustic lens 60, the ultrasonic transducers Tr are driven simultaneously, but the present disclosure is not limited thereto. For example, one channel may be implemented by the ultrasonic transducers Tr arranged in the Y direction and coupled by the lower electrodes 33A, and drive electrodes corresponding to the respective channels may be provided to independently drive the channels arranged in the X direction. In this case, the transmission direction of the ultrasonic waves can be controlled by delay-driving the channels. Further, each of the ultrasonic transducers Tr may be independently driven.

The ultrasonic element 30 as described above may further include a reinforcing plate to improve a substrate strength. When the reinforcing plate is provided, the reinforcing plate is provided on an opposite surface of the vibrating plate 32 from the element substrate 31. At this time, a spacer (bonding layer) is provided in a region other than the vibrating portions 32A to ensure a vibration space of each vibrating portion 32A, and the reinforcing plate is bonded via the spacer.

In the example illustrated in FIG. 4, the vibrating plate 32 is divided into the vibrating portions 32A by providing the substrate openings 31A in the element substrate 31. A wall formed of, for example, a resin material may be formed between the reinforcing plate and the vibrating plate 32, and the vibrating plate 32 may be divided into the vibrating portions 32A by the wall. In this case, the element substrate 31 may not be provided.

A surface of the vibrating plate 32 on the +Z side serves as a transmission and reception surface 30A (transmission surface of the present disclosure) to which the ultrasonic waves are transmitted to the +Z side by driving the piezoelectric element 33.

As illustrated in FIGS. 1 and 2, the housing 20 has a substantially rectangular parallelepiped shape and has an adhesive surface 21 that faces the living body. A window 22 is provided in the adhesive surface 21, and the transmission and reception surface 30A of the ultrasonic element 30 is exposed from the window 22. Here, in the embodiment, an example is described in which the adhesive surface 21 of the housing 20 is in the same plane as the transmission and reception surface 30A.

The housing 20 of the embodiment further includes a fixing portion 24 that assists fixing of the fixing sheet 50 when the fixing sheet 50 is attached.

In the embodiment, as illustrated in FIG. 1, the fixing portion 24 includes an engaging projection 241 and an engaging recess 55. The engaging projection 241 is provided at a side surface 23 intersecting with the adhesive surface 21 of the housing 20 and protrudes outward. The engaging recess 55 is a recess having substantially the same size as the engaging projection 241, and is provided at a base material 70 of the fixing sheet 50. The fixing sheet 50 is fixed to the housing 20 by a second adhesive layer 54 to be described later, and the fixing portion 24 assists the fixing by the second adhesive layer 54 and prevents peeling of the fixing sheet 50. Further, the fixing portion 24 may function as an alignment portion. For example, by engaging the engaging projection 241 with the engaging recess 55, the acoustic lens 60 to be described later may be positioned at a position facing the transmission and reception surface 30A of the ultrasonic element 30.

### Configuration of Fixing Sheet 50

When the ultrasonic probe 1 of the embodiment is fixed to the living body, the fixing sheet 50 is attached to the adhesive surface 21 of the ultrasonic device 10, and the fixing sheet 50 is attached and fixed to the living body. The fixing sheet 50 may be attached and fixed only to the adhesive surface 21 of the ultrasonic device 10, or may be attached from the adhesive surface 21 of the housing 20 to the side surface of the housing 20 as illustrated in FIG. 1.

The fixing sheet 50 includes the acoustic lens 60 and the base material 70. A surface of the fixing sheet 50 that faces the living body is a first surface 51, and an opposite surface, that is, a surface facing the ultrasonic device 10 is a second surface 52. Surfaces of the acoustic lens 60 and the base material 70 on the −Z side are in the same plane, and constitute the second surface 52 of the fixing sheet 50.

When the fixing sheet 50 is attached to the adhesive surface 21, the acoustic lens 60 is disposed at a position facing the transmission and reception surface 30A. The acoustic lens 60 is an element that changes the transmission direction of the ultrasonic waves transmitted from the transmission and reception surface 30A. For example, in the embodiment, the ultrasonic waves are converged to a predetermined depth position by the acoustic lens 60 formed in a cylindrical shape. Alternatively, a plurality of the fixing sheets 50 each having a different curvature of the acoustic lens 60 may be prepared, and the fixing sheet 50 including the acoustic lens 60 corresponding to a target depth of the living body may be attached to the ultrasonic device 10.

The base material 70 constitutes the fixing sheet 50 together with the acoustic lens 60. The base material 70 has flexibility and is deformable according to a shape of a living body surface or the ultrasonic probe. For example, in the example illustrated in FIG. 1, when the fixing sheet 50 is fixed to the ultrasonic device 10, the base material 70 having the flexibility is curved (bent) from the adhesive surface 21 to the side surface 23, and the fixing sheet 50 is made to follow a surface of the ultrasonic device 10.

In the embodiment, the base material 70 is provided to cover the entire acoustic lens 60 and the entire adhesive surface 21, and an opposite surface (that is, a +Z side surface) of the base material 70 from the ultrasonic device 10 serves as the first surface 51 to be attached to the living body. The base material 70 has an acoustic impedance different from that of the acoustic lens 60. For example, a sound speed in the acoustic lens 60 is slower than a sound speed in the base material 70.

Further, the base material 70 is provided with the engaging recess 55 constituting the fixing portion 24 as described above.

A first adhesive layer 53 (first fixing portion) for fixing the fixing sheet 50 to the living body is provided at the first surface 51 of the fixing sheet 50. The second adhesive layer 54 (second fixing portion) for fixing the fixing sheet 50 to the ultrasonic device 10 is provided at the second surface 52 of the fixing sheet 50.

The first adhesive layer 53 is implemented by an adhesive member that detachably fixes the fixing sheet 50 to the living body. In the embodiment, the base material 70 is provided to cover the entire +Z side of the acoustic lens 60, and the first adhesive layer 53 is provided to cover the entire base material 70 on the +Z side.

The second adhesive layer 54 is implemented by an adhesive member that detachably fixes the fixing sheet 50 to the ultrasonic device 10. The second adhesive layer 54 is provided to cover the entire second surface 52 which is the surface of the acoustic lens 60 and the base material 70 on the −Z side.

Here, the acoustic lens 60, the base material 70, the first adhesive layer 53, and the second adhesive layer 54 each have a difference in acoustic impedance with the living body less than a predetermined threshold. The first adhesive layer 53 and the second adhesive layer 54 may have the same acoustic impedance or different acoustic impedances. When the acoustic impedances are different, the first adhesive layer 53 is formed of a material having an acoustic impedance close to that of the living body and the base material 70, and the second adhesive layer 54 is formed of a material having an acoustic impedance close to that of the acoustic lens 60. Here, the description of “a difference in acoustic impedance less than a predetermined threshold” between the materials means that a sound intensity reflectance RI is less than a predetermined value. The “predetermined value” is, for example, an allowable value for transmitting ultrasonic waves of a strength corresponding to predetermined ultrasonic processing (for example, display processing of an internal tomographic image, or ultrasonic treatment of a target trunk) on the measurement target at a predetermined depth, and can be appropriately set according to a measurement depth and a content of the ultrasonic processing.

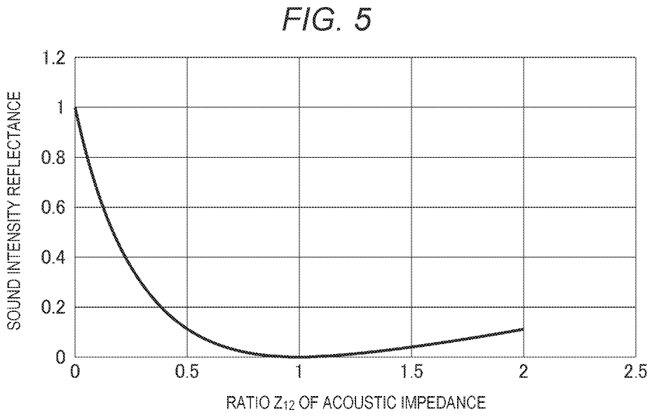

Here, when an acoustic impedance of a medium I is Z₁ and an acoustic impedance of a medium II is Z₂, the sound intensity reflectance RI when the ultrasonic waves are incident on the medium II from the medium I is represented by RI=(1−Z₁₂)²/(1+Z₁₂)². FIG. 5 is a diagram illustrating a correlation between a ratio Z₁₂(=Z₂/Z₁) of the acoustic impedance and the sound intensity reflectance RI.

In the embodiment, the sound intensity reflectance RI is set to be less than 5%. In this case, as illustrated in FIG. 5, the material for each part may be selected such that the ratio Z₁₂ of the acoustic impedance satisfies 0.65<Z₁₂<1.55. For example, when the living body is a target, the base material 70 is silicone, the acoustic lens 60 is silicone rubber, and the first adhesive layer 53 and the second adhesive layer 54 are silicone-based adhesives. The acoustic lens 60 and the base material 70 need to have different acoustic impedances. However, the first adhesive layer 53 and the second adhesive layer 54 may have the same acoustic impedance as that of the acoustic lens 60, the base material 70, or the living body.

### Fixing of Ultrasonic Probe 1 to Living Body

A protective sheet (not illustrated) is attached to each of the first adhesive layer 53 and the second adhesive layer 54 in a state in which the fixing sheet 50 as described above is not attached to the ultrasonic device 10. The protective sheet is a sheet for preventing adhesion of foreign matters to the first adhesive layer 53 and the second adhesive layer 54 before the ultrasonic probe 1 is used. When the ultrasonic probe 1 is used, the protective sheet is peeled off from the first adhesive layer 53 and the second adhesive layer 54.

First, the protective sheet attached to the second adhesive layer 54 is peeled off from the fixing sheet 50, and the second adhesive layer 54 is brought into contact with the adhesive surface 21 of the housing 20 to attach the fixing sheet 50.

At this time, the fixing portion 24 may be used to align the fixing sheet 50 with the adhesive surface 21. Accordingly, the acoustic lens 60 can be appropriately positioned at the transmission and reception surface 30A of the ultrasonic element 30.

Then, the protective sheet attached to the first adhesive layer 53 is peeled off from the fixing sheet 50, and the first adhesive layer 53 is brought into contact with the living body to fix the fixing sheet 50 to the living body. Accordingly, the ultrasonic probe 1 is fixed to the living body.

### Functions and Effects of Embodiment

The ultrasonic probe 1 of the embodiment includes the ultrasonic device 10 that transmits the ultrasonic waves, and the fixing sheet 50 provided at the transmission and reception surface 30A of the ultrasonic device 10 to come into contact with the living body that is the measurement target. The fixing sheet 50 includes the acoustic lens 60 that guides the ultrasonic waves transmitted from the ultrasonic device 10 in a predetermined direction, the base material 70 that covers at least a part of the acoustic lens 60 and has a difference in acoustic impedance with the living body less than a predetermined threshold, and the first adhesive layer 53 that is provided at the first surface 51 of the base material 70 and detachably fixes the ultrasonic device 10 to the living body. The first surface 51 faces the living body.

In such an embodiment, the fixing sheet 50 is interposed between the ultrasonic device 10 and the living body, and the acoustic lens 60, the base material 70, and the first adhesive layer 53 constituting the fixing sheet 50 are configured such that a difference in acoustic impedance with the living body is close, that is, the sound intensity reflectance RI is less than 5%. Therefore, inconvenience in which the ultrasonic waves transmitted from the ultrasonic probe 1 are inhibited by the fixing sheet 50 interposed between the living body and the ultrasonic probe 1 is prevented, and the ultrasonic waves can be transmitted to the living body with a high propagation efficiency.

When the ultrasonic device 10 is repeatedly used for the living body in a medical site or the like, the ultrasonic device 10 needs to be sterilized to prevent infection. In the sterilization, a sterilization device is usually used, and in the sterilization device, after the ultrasonic probe 1 is put into a sterilization chamber, an inside of the chamber is depressurized to be evacuated, and the sterilization is performed by filling the chamber with a sterilization gas. At this time, in the embodiment, the ultrasonic probe 1 is put into the sterilization chamber in a state in which the fixing sheet 50 is attached to the ultrasonic device 10. Therefore, the vibrating plate 32 exposed from the housing 20 is covered with the fixing sheet 50 when the chamber is depressurized, a stress on the vibrating plate 32 caused by the depressurization can be reduced, and damage to the ultrasonic device 10 can be prevented.

In the embodiment, the fixing sheet 50 further includes the second adhesive layer 54 provided at the second surface 52 facing the ultrasonic device 10 and detachably fixing the fixing sheet 50 to the ultrasonic device 10.

Therefore, the fixing sheet 50 attached to the ultrasonic device 10 can be replaced. That is, the fixing sheet 50 that comes into contact with the living body can be replaced every time the ultrasonic probe 1 is used, and the ultrasonic probe 1 can be attached to the living body hygienically.

Further, by preparing the plurality of fixing sheets 50 each having a different transmission direction of the ultrasonic waves of the acoustic lens 60, it is possible to select the fixing sheet 50 including the acoustic lens 60 corresponding to the target depth in the living body and a usage purpose of the ultrasonic probe 1.

In the embodiment, the base material 70 covers the entire acoustic lens 60.

Accordingly, in the embodiment, the first surface 51 of the base material 70 on a living body side can be formed into a flat surface. In such a configuration, even when an acoustic gel or the like is not used, the ultrasonic waves can be transmitted to the living body with a high propagation efficiency without air bubbles being mixed between the acoustic lens 60 and the base material 70.

In the embodiment, the first adhesive layer 53 covers the entire first surface 51 of the base material 70.

As described above, when the entire acoustic lens 60 is covered with the base material 70, the first surface 51 of the base material 70 on the living body side can be made into a flat surface. By providing the first adhesive layer 53 to cover the entire first surface 51, air bubbles can be prevented from being mixed between the fixing sheet 50 and the living body when the fixing sheet 50 is fixed to the living body.

In the embodiment, the ultrasonic device 10 includes the ultrasonic element 30 that has the transmission and reception surface 30A and transmits the ultrasonic waves from the transmission and reception surface 30A, and the housing 20 accommodating the ultrasonic element 30. The ultrasonic element 30 includes the vibrating plate 32 having the transmission and reception surface 30A and the piezoelectric element 33 provided at the vibrating plate 32. The piezoelectric element 33 is driven to vibrate the vibrating plate 32 to transmit the ultrasonic waves from the transmission and reception surface 30A. The housing 20 includes the window 22 exposing the transmission and reception surface 30A to the adhesive surface 21 facing the fixing sheet 50, and further includes the fixing portion 24 that engages the fixing sheet 50 with the housing 20. In the embodiment, the fixing sheet 50 is fixed to the housing 20 by the second adhesive layer 54, and the fixing portion 24 can be used to assist in fixing by the second adhesive layer 54 and prevent the peeling of the fixing sheet 50.

Further, in the embodiment, the fixing portion 24 may function as the alignment portion of the present disclosure. Accordingly, when the fixing sheet 50 is fixed to the ultrasonic device 10, a position of the fixing sheet 50 with respect to the ultrasonic device 10 can be appropriately adjusted by the fixing portion 24. That is, the fixing sheet 50 can be positioned at the ultrasonic device 10 such that the acoustic lens 60 faces the transmission and reception surface 30A exposed from the window 22.

### Second Embodiment

Next, a second embodiment will be described.

In the first embodiment, the base material 70 of the fixing sheet 50 covers the entire acoustic lens 60. The second embodiment differs from the first embodiment in that a part of the acoustic lens 60 is exposed.

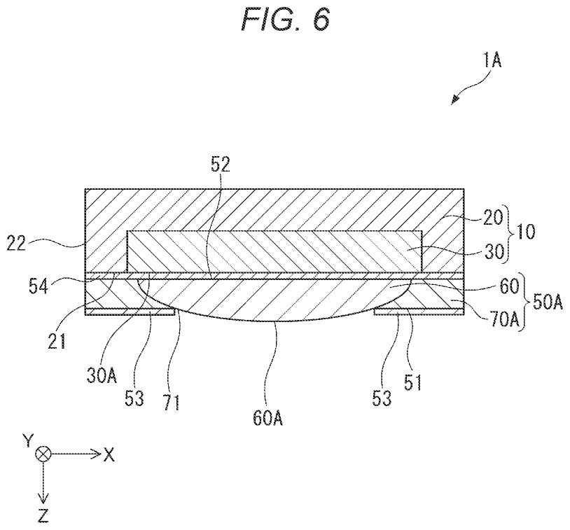

FIG. 6 is a cross-sectional view schematically illustrating a schematic configuration of an ultrasonic probe 1A according to the second embodiment.

In the following description, the same components are denoted by the same reference numerals, and description thereof is omitted or simplified.

As in the first embodiment, the ultrasonic probe 1A of the embodiment includes the ultrasonic device 10 and a fixing sheet 50A. A configuration of the ultrasonic device 10 is the same as that of the first embodiment.

In contrast, the fixing sheet 50A includes the acoustic lens 60 and a base material 70A. The base material 70A includes an opening window 71.

The opening window 71 exposes a predetermined range of the acoustic lens 60. That is, in the embodiment, the base material 70A does not cover the entire acoustic lens 60, and the acoustic lens 60 is exposed to the +Z side from the opening window 71.

For example, in FIG. 6, the acoustic lens 60 has a cylindrical shape and is formed in a convex shape in which a central axis along a Y axis is a top portion 60A as in the first embodiment. The opening window 71 exposes a range of a predetermined dimension in a ±X direction about the top portion 60A of the acoustic lens 60.

The first adhesive layer 53 is formed at a surface of the base material 70A on the +Z side. That is, in the example of FIG. 6, the first adhesive layer 53 is not provided at a surface of the acoustic lens 60 on the +Z side, and the acoustic lens 60 directly comes into contact with the living body in a predetermined range about the top portion 60A.

In the ultrasonic probe 1A, as in the first embodiment, after the fixing sheet 50A is fixed to the ultrasonic device 10, the first adhesive layer 53 is exposed. Then, the opening window 71 of the first adhesive layer 53 is filled with an acoustic gel, and the first adhesive layer 53 is attached to the living body. Accordingly, inconvenience in which an air layer is formed in a space between the opening window 71 and the acoustic lens 60 can be prevented, and the ultrasonic waves can be transmitted from the acoustic lens 60 to the living body without passing through the base material 70A and the first adhesive layer 53. The filling of the acoustic gel into the opening window 71 is not necessary, and for example, when the ultrasonic probe 1A is directly attached to body tissue, since body fluids take the place of the acoustic gel, there is no need to use the acoustic gel.

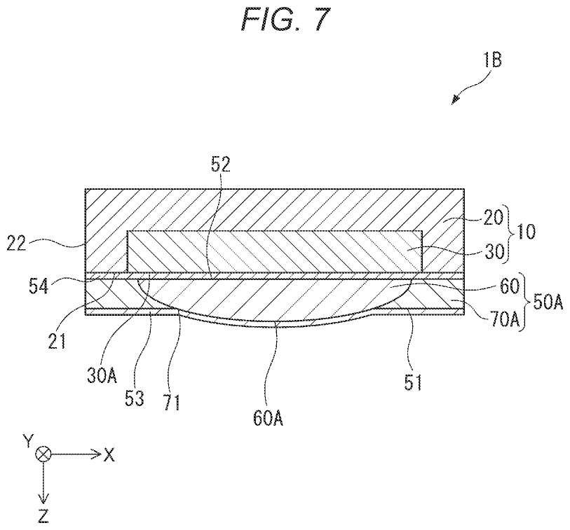

FIG. 7 is a cross-sectional view schematically illustrating a configuration of another ultrasonic probe 1B according to the second embodiment.

As illustrated in FIG. 7, the first adhesive layer 53 may cover the entire base material 70A and acoustic lens 60 on the +Z side. In this case, there is no need to fill a space between the living body and the fixing sheet 50A with an adhesive gel.

### Functions and Effects of Embodiment

In the embodiment, the acoustic lens 60 has a cylindrical shape, and the base material 70A includes the opening window 71 exposing a predetermined range of the acoustic lens 60.

Accordingly, a part of the acoustic lens 60 is exposed from the opening window 71 to the living body side. Therefore, since the acoustic lens 60 is not covered by the base material 70A, the ultrasonic waves can be propagated into the living body more efficiently.

### Third Embodiment

Next, a third embodiment will be described.

In the first embodiment, the first surface 51, that is, a surface of the base material 70 on the +Z side is a flat surface. The third embodiment differs from the first embodiment in that a recess is provided.

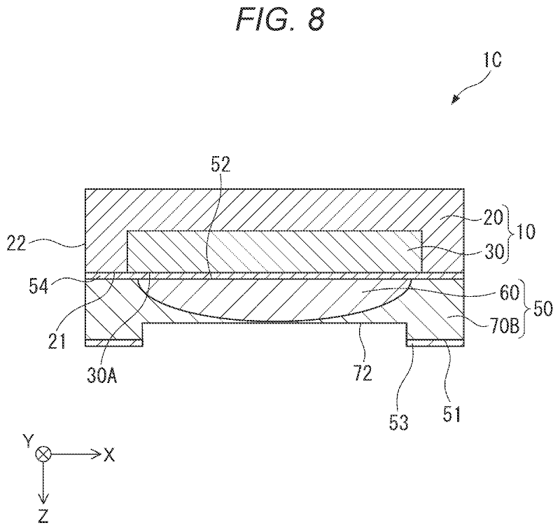

FIG. 8 is a cross-sectional view schematically illustrating a schematic configuration of an ultrasonic probe 1C according to the third embodiment.

In the embodiment, as illustrated in FIG. 8, a base material 70B is provided with a recess 72 that is recessed toward the acoustic lens 60 at a position overlapping the acoustic lens 60 when viewed from the Z direction at the first surface 51 on the +Z side.

The recess 72 is a filling position of an acoustic gel. That is, in the embodiment, when the ultrasonic probe 1C is fixed to the living body, the fixing sheet 50 is fixed to the living body by the first adhesive layer 53 after the recess 72 is filled with the acoustic gel. In this case, as compared with FIG. 6 of the second embodiment, by providing the recess 72, the acoustic gel can be held, and inconvenience in which the acoustic gel flows out can be prevented.

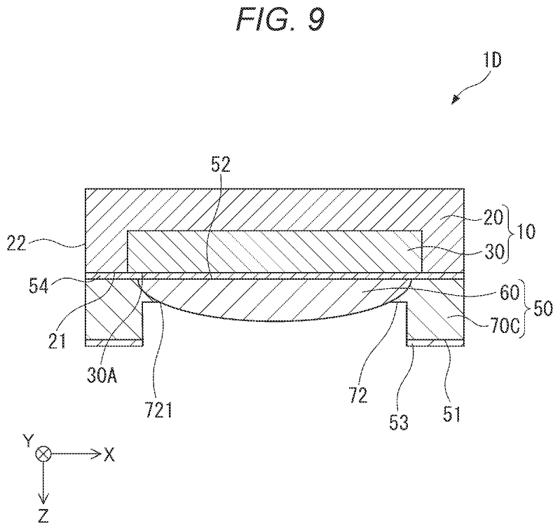

FIG. 9 is a cross-sectional view schematically illustrating a configuration of another ultrasonic probe 1D according to the third embodiment.

In the example illustrated in FIG. 8, the base material 70B covers the entire acoustic lens 60, and covers the acoustic lens 60 at a position at which a bottom portion of the recess 72 overlaps the acoustic lens 60 when viewed from the Z direction.

In contrast, as in a base material 70C illustrated in FIG. 9, a bottom portion opening window 721 is provided at the bottom portion of the recess 72, and the acoustic lens 60 is exposed from the recess 72 as in the second embodiment.

### Functions and Effects of Embodiment

In the embodiment, when viewed from the Z direction, each of the base materials 70B and 70C includes the recess 72 that is recessed from a side that faces the living body toward the acoustic lens 60 in a region overlapping the acoustic lens 60. The first adhesive layer 53 covers a portion of each of the base materials 70B and 70C where the recess 72 is not provided.

Accordingly, when the fixing sheet 50 is attached to the living body, the recess 72 is filled with the acoustic gel to fix the ultrasonic probe 1C to the living body. Therefore, there is no need to consider an acoustic impedance of the first adhesive layer 53, and a degree of freedom in designing the fixing sheet 50 can be improved.

### Modifications

The present disclosure is not limited to the embodiments and modifications described above. The present disclosure includes modifications, improvements, and configurations obtained by appropriately combining the embodiments within a scope where an object of the present disclosure can be achieved.

### Modification 1

In the above-described embodiments, an example is described in which the adhesive surface 21 of the housing 20 and the transmission and reception surface 30A of the ultrasonic element 30 are in the same plane, and the present disclosure is not limited thereto.

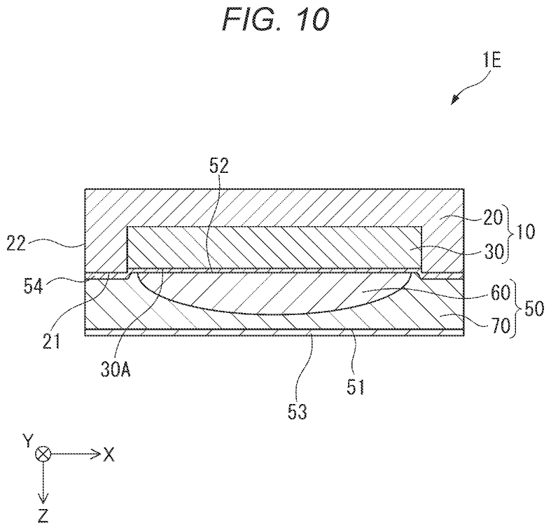

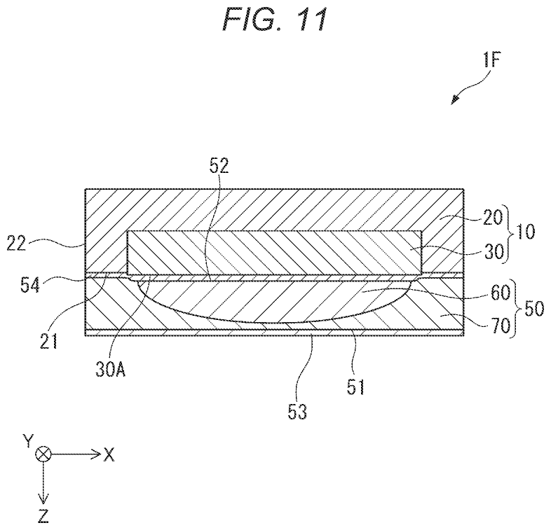

FIG. 10 is a cross-sectional view schematically illustrating a schematic configuration of an ultrasonic probe 1E according to Modification 1. FIG. 11 is a cross-sectional view schematically illustrating a schematic configuration of another ultrasonic probe 1F according to Modification 1.

For example, as illustrated in FIG. 10, the transmission and reception surface 30A may be located far from the living body and have a recess shape with respect to the adhesive surface 21. Further, as illustrated in FIG. 11, the transmission and reception surface 30A may protrude toward the living body with respect to the adhesive surface 21. In either case, when a step between the adhesive surface 21 and the transmission and reception surface 30A is within a half value of a thickness of the second adhesive layer 54, as illustrated in FIGS. 10 and 11, the fixing sheet 50 can be attached to the ultrasonic device 10 while preventing entry of air bubbles between the ultrasonic device 10 and the fixing sheet 50 due to deformation of the second adhesive layer 54.

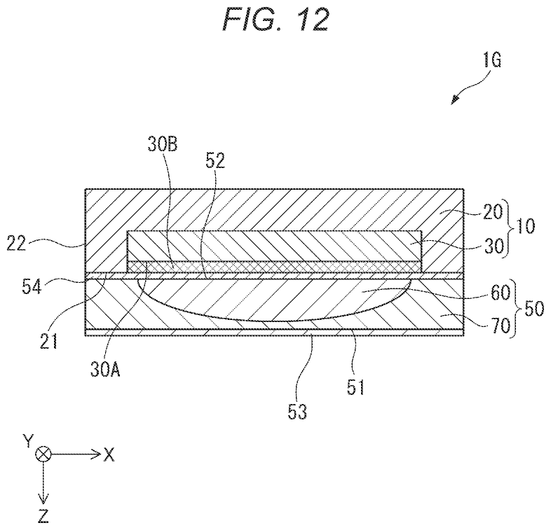

FIG. 12 is a cross-sectional view schematically illustrating a schematic configuration of still another ultrasonic probe 1G according to Modification 1.

As illustrated in FIG. 12, when the transmission and reception surface 30A is located at a position away from the living body by a distance equal to or greater than the thickness of the second adhesive layer 54 with respect to the adhesive surface 21, a step portion between the transmission and reception surface 30A and the adhesive surface 21 is filled with a filler 30B. Accordingly, surfaces of the adhesive surface 21 and the filler 30B on the +Z side are in the same plane, and air bubbles can be prevented from being mixed when the second adhesive layer 54 is attached.

### Modification 2

In the above-described embodiments, a configuration is described in which in the ultrasonic element 30, the piezoelectric elements 33 are disposed at the vibrating plate 32, and the ultrasonic waves are transmitted by vibrating the vibrating portion 32A of the vibrating plate 32 by the piezoelectric elements 33. In contrast, the ultrasonic element 30 may have a configuration in which a voltage is applied to a piezoelectric body to vibrate the piezoelectric body itself.

### Modification 3

In the above-described embodiments, an example is described in which the acoustic lens 60 has a cylindrical shape, and the ultrasonic waves transmitted from the transmission and reception surface 30A are converged to a predetermined depth position of the living body. In contrast, the acoustic lens 60 may have a configuration in which the ultrasonic waves are diverged, that is, may be provided as a recessed-shaped acoustic lens. In other words, the ultrasonic waves transmitted from an inside of the living body is converged to the ultrasonic element 30.

In this case, the ultrasonic probe may function as a probe for receiving the ultrasonic waves.

### Modification 4

In the above-described embodiments, an example is described in which the fixing sheet 50 is detachably fixed to the ultrasonic device 10 by the second adhesive layer 54, and the present disclosure is not limited thereto.

For example, the fixing sheet 50 may be firmly bonded to the ultrasonic device 10 by a bonding layer or the like.

### Modification 5

In the above-described embodiments, description is made in which the fixing portion 24 may function as an alignment portion for positioning the fixing sheet 50 with respect to the housing 20. In contrast, an alignment portion different from the fixing portion 24 may be further provided.

That is, in the above-described embodiments, a configuration example in which the fixing sheet 50 is attached from the adhesive surface 21 of the housing 20 to the side surface 23 and is engaged with the fixing portion 24 is stated. The fixing sheet 50 may be attached only to the adhesive surface 21 of the housing 20. In this case, an alignment portion for positioning the housing 20 and the fixing sheet 50 may be provided at the adhesive surface 21.

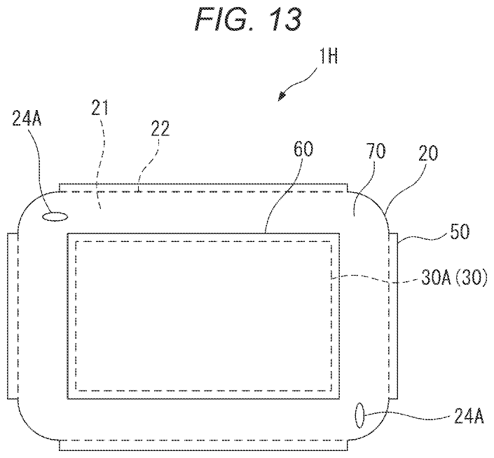

FIG. 13 is a view of an ultrasonic probe 1H according to Modification 5 as viewed from the Z direction.

For example, in the ultrasonic probe 1H illustrated in FIG. 13, alignment portions 24A are provided at the adhesive surface 21. In this case, an engaging projection may be provided at any one of the adhesive surface 21 and the second surface 52 of the base material 70 of the fixing sheet 50, and an engaging recess may be provided at the other.

The alignment portion 24A is not limited to a configuration in which the engaging projection is engaged with the engaging recess. For example, when the base material 70 is formed of a transparent member, the housing 20 may be provided with a first alignment mark, the fixing sheet 50 may be provided with a second alignment mark, and when the acoustic lens 60 is disposed at a position facing the transmission and reception surface 30A, the first alignment mark and the second alignment mark may coincide with each other. In this case, the fixing sheet 50 is positioned such that the first alignment mark and the second alignment mark coincide with each other by visual observation. Alternatively, the fixing sheet 50 may be positioned at the ultrasonic device 10 by any method.

### Summary of Present Disclosure

An ultrasonic probe of an aspect according to the present disclosure includes: an ultrasonic device configured to transmit an ultrasonic wave; and an acoustic sheet provided at a transmission surface of the ultrasonic wave of the ultrasonic device and configured to come into contact with a measurement target, in which the acoustic sheet includes an acoustic lens configured to guide the ultrasonic wave output from the transmission surface in a predetermined direction, a base material covering at least a part of the acoustic lens and having a difference in acoustic impedance with the measurement target less than a predetermined threshold, and a first fixing portion provided at least at a first surface of the base material and configured to detachably fix the acoustic sheet to the measurement target, the first surface being configured to face the measurement target.

In the present aspect, the acoustic sheet is interposed between the ultrasonic device and the measurement target, and the acoustic lens, the base material, and the first fixing portion constituting the acoustic sheet each have a close difference in acoustic impedance with the measurement target. The phrase “the difference in acoustic impedance is close” means that, for example, the sound intensity reflectance is less than a predetermined value. Accordingly, inconvenience is prevented in which the ultrasonic waves transmitted and received by the ultrasonic probe are inhibited by the acoustic sheet interposed between the ultrasonic probe and the measurement target, and the ultrasonic waves can be transmitted to the measurement target with a high propagation efficiency.

In the ultrasonic probe of the present aspect, the acoustic sheet may include a second fixing portion provided at a second surface and configured to detachably fix the acoustic sheet to the ultrasonic device, the second surface being configured to face the ultrasonic device.

Accordingly, the acoustic sheet attached to the ultrasonic device can be replaced. That is, it is possible to replace the acoustic sheet that comes into contact with the measurement target every time the ultrasonic probe is used, and when a living body is a target as the measurement target, the ultrasonic probe can be used hygienically.

Further, when a plurality of the acoustic sheets each having a different transmission direction of the ultrasonic waves of the acoustic lens are prepared, it is possible to select the acoustic sheet including the acoustic lens corresponding to a target depth in the measurement target and a usage purpose of the ultrasonic probe. That is, one ultrasonic probe can be used for measurement at various measurement depths.

In the ultrasonic probe of the present aspect, the base material may cover the entire acoustic lens.

Accordingly, the first surface of the base material on a measurement target side can be formed into a flat surface. In such a configuration, even when an acoustic gel or the like is not used, the ultrasonic waves can be transmitted to the measurement target with a high propagation efficiency without air bubbles being mixed between the acoustic lens and the base material.

In the ultrasonic probe of the present aspect, the first fixing portion may cover the entire first surface of the base material.

As in the aspect described above, when the entire acoustic lens is covered with the base material, the first surface of the base material on the measurement target side can be made into a flat surface. Further, by providing the first fixing portion to cover the entire first surface, air bubbles can be better prevented from being mixed between the acoustic sheet and the measurement target when the acoustic sheet is fixed to the measurement target.

In the ultrasonic probe of the present aspect, when viewed from a direction orthogonal to the transmission and reception surface, the base material may include a recess that is recessed from a side configured to face the measurement target toward the acoustic lens in a region overlapping the acoustic lens, and the first fixing portion may cover a portion of the base material in which the recess is not provided.

In the present aspect, the ultrasonic probe can be fixed to the measurement target by filling the recess with an acoustic gel having an acoustic impedance close to that of the measurement target. In this case, there is no need to consider a difference in acoustic impedance between the first fixing portion and the measurement target, and a degree of freedom in selecting a material for the first fixing portion can be improved.

In the ultrasonic probe of the present aspect, the acoustic lens may have a cylindrical shape, and the base material may include an opening window exposing a predetermined range of the acoustic lens.

In the present aspect, the acoustic lens having a cylindrical shape may be pressed against the measurement target to fix the ultrasonic probe, the ultrasonic probe may be fixed by filling a space between the acoustic lens and the measurement target with the acoustic gel, and the first fixing portion may be provided to cover the acoustic lens. In any case, there is no need to consider a difference in acoustic impedance between the measurement target and the base material, and the degree of freedom in selecting a material for the base material can be improved.

In the ultrasonic probe of the present aspect, the housing may include an alignment portion configured to position the acoustic sheet such that the acoustic lens is disposed at a position configured to face the transmission surface.

Accordingly, the acoustic sheet can be properly positioned and fixed to the ultrasonic device.

In the ultrasonic probe of the present aspect, the ultrasonic device may include an ultrasonic element having the transmission surface and configured to transmit the ultrasonic wave from the transmission surface, and a housing accommodating the ultrasonic element, and the ultrasonic element may include a vibrating plate having the transmission surface and a piezoelectric element provided at the vibrating plate, and transmit the ultrasonic wave from the transmission surface by driving the piezoelectric element and vibrating the vibrating plate.

As in the present aspect, in a configuration in which the ultrasonic device includes the vibrating plate and the piezoelectric element above the vibrating plate, a thin ultrasonic device can be implemented, and the ultrasonic probe can be made smaller. Further, by adopting a configuration in which the transmission surface of the vibrating plate is exposed from the window, the ultrasonic waves can be directly propagated to the acoustic lens provided corresponding to the window, and strong ultrasonic waves can be transmitted with a high propagation efficiency.

What is claimed is:

1. An ultrasonic probe comprising:
an ultrasonic device configured to transmit an ultrasonic wave; and
an acoustic sheet provided at a transmission surface of the ultrasonic wave of the ultrasonic device, wherein
the acoustic sheet includes
an acoustic lens configured to guide the ultrasonic wave output from the transmission surface in a predetermined direction,
a base material covering at least a part of the acoustic lens and having a difference in acoustic impedance with a measurement target less than a predetermined threshold, and
a first fixing portion provided at least at a first surface of the base material and configured to detachably fix the acoustic sheet to the measurement target, the first surface being configured to face the measurement target.

2. The ultrasonic probe according to claim 1, wherein
the acoustic sheet includes a second fixing portion provided at a second surface and configured to detachably fix the acoustic sheet to the ultrasonic device, the second surface being configured to face the ultrasonic device.

3. The ultrasonic probe according to claim 1, wherein
the base material covers the entire acoustic lens.

4. The ultrasonic probe according to claim 3, wherein
the first fixing portion covers the entire first surface of the base material.

5. The ultrasonic probe according to claim 3, wherein
when viewed from a direction orthogonal to the transmission surface, the base material includes a recess that is recessed from a side configured to face the measurement target toward the acoustic lens in a region overlapping the acoustic lens, and
the first fixing portion covers a portion of the base material in which the recess is not provided.

6. The ultrasonic probe according to claim 1, wherein
the acoustic lens has a cylindrical shape, and
the base material includes an opening window exposing a predetermined range of the acoustic lens.

7. The ultrasonic probe according to claim 2, wherein
the ultrasonic device includes an alignment portion configured to position the acoustic sheet such that the acoustic lens is disposed at a position configured to face the transmission surface.

8. The ultrasonic probe according to claim 1, wherein
the ultrasonic device includes
an ultrasonic element having the transmission surface and configured to transmit the ultrasonic wave from the transmission surface, and
a housing accommodating the ultrasonic element, and
the ultrasonic element includes a vibrating plate having the transmission surface and a piezoelectric element provided at the vibrating plate, and transmits the ultrasonic wave from the transmission surface by driving the piezoelectric element and vibrating the vibrating plate.
